(19) United States
(12) Patent Application Publication
(10) Pub. No.: US 2025/0195441 A1
(43) Pub. Date: Jun. 19, 2025

(54) GASTRORETENTIVE FORMULATIONS CONTAINING PROTEIN OR PEPTIDE

(71) Applicant: APET HOLDING B.V., IJsselstein (NL)

(72) Inventors: Lekhram CHANGOER, IJsselstein (NL); Hendrik Jan Cornelis MEIJERINK, Wespelaar (BE)

(73) Assignee: APET HOLDING B.V., IJsselstein (NL)

(21) Appl. No.: 18/843,712

(22) PCT Filed: Mar. 6, 2023
(86) PCT No.: PCT/EP2023/055592

§ 371 Date: Sep. 4, 2024

(30) Foreign Application Priority Data
Mar. 4, 2022 (EP) 22160189.1

(51) Int. Cl. A61K 9/48; A61K 9/00

(52) U.S. Cl. CPC A61K 9/4891; A61K 9/0065; A61K 9/4825; A61K 9/4866

(57) ABSTRACT

The invention relates to the design of floating drug delivery systems (FDDS) in such way that proteins and/or peptides, loaded on the FDDS, can be delivered for a prolonged time to the upper part of the gastrointestinal tract. The long-acting formulation according to the invention hence overcomes the problem in the prior art to continuously deliver a protein and/or peptide to the stomach and/or proximal part of the intestinal tract for an extended period of time so that these molecules can exert their function or can be taken up at the site of interest. Furthermore, the procedure of manufacturing the floating drug delivery system of the invention is simple and straightforward, and therefore economically attractive.

### FIELD OF THE INVENTION

The present invention relates to the field of oral gastro-retentive delivery systems, in particular floating drug delivery systems for delivery of proteins and peptides, and the uses thereof in therapy.

### BACKGROUND OF THE INVENTION

Oral administration of an active pharmaceutical or nutraceutical ingredient is the most preferable way of delivery. The oral route offers the advantages of self-administration with a high degree of customer and/or patient acceptability and compliance due to the simple and comfortable use and flexibility regarding dose strength and type of formulation. Numerous pharmaceuticals are active in the digestive tract, in which case oral administration is not just the most comfortable route but also the most effective one. Currently, more than 60% of the traditional small molecule pharmaceuticals available in the market are administered via the oral route.

Peptides and proteins offer several advantages as compared to conventional drugs. These include high activity, high specificity, low toxicity, and minimal nonspecific and drug-drug interactions. Peptides and proteins have become the preferred drugs in certain disease states such as enzyme deficiency, genetic and degenerative disease and protein-dysfunction. Developments in the field of biotechnology resulted in viable large-scale production of peptides, hormones and vaccines. At present there are more than 40 peptide and protein drugs in the market worldwide with approximately 270 peptides in clinical phase and 400 peptides in advanced preclinical phase testing.

Compared with traditional small molecule pharmaceuticals, peptides and proteins are considerably larger molecular entities with inherent physiochemical complexities, from their primary amino acid sequences through higher-order secondary and tertiary structures—and in some cases, quaternary elements such as subunit associations. Due to these physicochemical complexities formulation development for peptide and protein drugs is far more complex than for chemical compounds.

A first important factor in this regard is the generally low oral bioavailability of peptide and protein drugs, stemming mainly from pre-systemic enzymatic degradation and poor penetration across the intestinal membrane.

An equally important factor is the (potential for) degradation of peptides and proteins during the processes required to manufacture a suitable formulation, especially in the case of complex solid dosage units. Degradation events that can occur in formulation manufacturing (and/or storage) involve (but are not limited to) oxidation, photodegradation, disulfide scrambling, deamidation, aggregation, precipitation, dissociation, fragmentation.

Aggregation and precipitation of proteins or peptides is a particular concern in formulation manufacturing. It may be associated with decreased bioactivity and increased immunogenicity. The potential for aggregation is typically enhanced by exposure of a protein or peptide to liquid-air, liquid-solid, and even liquid-liquid interfaces. Mechanical stress, heating, freezing and thawing can also cause/enhance protein aggregation. Solution conditions such as protein concentration, pH, and ionic strength affect the tendency and rate of proteins and peptides to aggregate. The impact of aggregation on product potency varies and depends on the physiochemical attributes of each protein relative to its functional domains and the nature of the activity being measured. Because of the many physical and chemical manipulations required in formulation operations, aggregation of proteins and peptides poses a tremendous challenge in formulation development.

Unsurprisingly, to date, most of the commercially available proteins and peptides are delivered via intramuscular (IM), subcutaneous (SC), or intravenous (IV) injections using relatively simple liquid formulations.

The present invention generally aims to provide a solid controlled release formulation that can be used to deliver (functional) peptides and proteins via the oral route. One particular objective of the invention is to provide a suitable controlled release formulation for delivering locally acting peptides and proteins to the site of action in the digestive tract. Another aim of the invention is to provide a controlled release formulation that can serve as a versatile platform for targeting systemically active peptides and proteins to the site of absorption in the digestive tract. Another aim of the invention is to provide a method which enables the loading of high dosages, allowing therapeutically effective dosages to be delivered by once-a-day administration.

### SUMMARY OF THE INVENTION

The present invention is in part predicated on the surprising finding that a certain gastroretentive formulation is very suitable for the oral delivery of locally and/or systemically acting proteins and peptides.

In one aspect, the present invention provides a floating drug delivery system (FDDS), said FDDS comprising a particle having a hollow, gas-filled core bordered by a wall of at least one material selected from the group of aqueous soluble, erodible, disintegrating and biodegradable materials, preferably a polymer, said wall being surrounded by a coating comprising at least one protein and/or peptide.

The FDDS according to the invention can be produced by processes involving spray-application of the coating layers using processes that, surprisingly, proved to preserve most or all of the activity of the protein, as demonstrated in the appended examples using an active enzyme. The present invention hence provides a versatile, long-acting formulation to allow a continuous release of protein or peptide for an extended period of time so that these molecules can effectively be targeted to the site of action or absorption in the gastrointestinal tract.

It is another object of the present invention to provide a FDDS wherein the particle/capsule is provided with a low pH resistant subcoating. A FDDS according to this embodiment has the advantage that the penetration of acid water in the centre of the capsule is further avoided.

The floating drug delivery systems of the invention allow for incorporation of high loads of active ingredients, as will be apparent from the examples, and present a slow release profile of the protein and/or peptide in the stomach and proximal small intestine for at least 6 hours, allowing therapeutically effective dosages to be delivered by once-a-day administration.

These and other aspects of the invention and its preferred embodiments will be described in more detail and exemplified in the following sections.

### DETAILED DESCRIPTION OF THE INVENTION

A first aspect of the invention concerns a floating drug delivery system (FDDS), said FDDS comprising:
(i) a hollow particle, comprising a gas-filled and/or hollow core bordered by a wall of at least one material selected from the group of aqueous soluble, erodible, disintegrating and biodegradable materials, preferably a polymer; and
(ii) at least one coating surrounding said hollow particle comprising at least one protein and/or peptide in combination with at least one coating excipient.

The wall of the hollow particle is made of an aqueous soluble, erodible, disintegrating and/or biodegradable material, typically a polymer, such that the FDDS can disintegrate in the body without leaving any traces behind in the body. Suitable polymers that are aqueous soluble, erodible, disintegrating and/or biodegradable are well known in the art, and include gelatine and hydroxypropyl methylcellulose (HPMC). HPMC is also known in the art as hypromellose. The FDDS system can be produced using only excipients that are known to be safe for human or animal use and that are accepted by regulatory authorities.

As will be understood, the gas contained in the hollow particle is a non-toxic gas. Air is the preferred gas. Because of the gas-filled compartment, lacking any particulate matter or matrix components, the FDDS provided herein have a unique floating capacity and therefore very good gastric retention properties.

The shape and size of the hollow particle can vary. Of course, for oral administration purposes it is preferred that the particle can be swallowed. A preferred hollow particle is a conventional gastric erodible/soluble capsule, such as a gelatine capsule. Also encompassed are ‘vegetarian capsules’, which are typically produced from vegetable derived material, preferably comprising a cellulosic derivative such as a HPMC capsule, Vcaps®, DRcaps™ and enTRinsic™ drug delivery technology capsules. Other suitable vegetarian capsules include those made from pullulan which is made through natural fermentation of tapioca (Plantcaps®). Both soft and hard shell capsules are encompassed. The particle can be a single or a multi-particulate capsule. In one embodiment, the invention provides a FDDS comprising a capsule having a hollow, gas-filled core bordered by a wall of at least one aqueous soluble, erodible, disintegrating or degradable material, typically a polymer, said wall being surrounded by a coating comprising at least one active ingredient. In view of gastric retention time, it is preferred that an oral gastro-retentive dosage form is as large as possible (to minimize passage through the pylorus) yet sufficiently small to be swallowed. Preferably, a FDDS provided herein comprises an oblong shaped capsule having a length of at least 10 mm, preferably at least 14 mm, more preferably at least 16 mm, most preferably at least 19 mm, and/or a diameter of at least 5 mm preferably at least 6 mm, more preferred at least 7, most preferred at least 8 mm. Suitable capsules include those referred to in the art as Type 5, 4, 3, 2, 2el, 1, 1el, 0, 0el, 00, 00el or 000 capsules. Alternatively, wide body capsules (BDCaps®) may be used. These capsules are referred to in the art as E, D, C, B, A, AA, AAel or AAA. Preferably, gelatine capsule type 4 is used.

As defined here above, the FDDS of the invention comprises at least one coating, surrounding the hollow particle. Said at least one coating comprises the peptide or protein.

It will be understood that the expression “protein” refers to any oligopeptide, polypeptide, gene product or expression product. The term “protein” encompasses naturally occurring or synthetic molecules, with or without the presence of modifications.

It will also be understood that the expression “peptide” refers to any peptide, independent from the presence of naturally and/or non-naturally occurring modifications. A peptide may comprise >3, >4, 5 to 10 or 5 to 20 or 5 to 30 or 5 to 50 amino acids.

Furthermore, it will be understood that the present invention typically entails the incorporation of active or functional peptides or proteins in the FDDS.

In one embodiment of the invention, the FDDS comprises a protein or peptide that acts locally in the stomach, the intestine and/or the colon.

In an embodiment of the invention, the protein is a digestive enzyme. As is understood by those skilled in the art, certain digestive enzymes are administered to human or animal subjects to treat pathologies or conditions associated with insufficient endogenous production of said enzyme. Additionally, it has become increasingly common to administer digestive enzymes to animal, in particular feedstock, to increase feed conversion, which may have therapeutic, economic or even environmental benefits. Digestive enzymes that can beneficially be formulated in the FDDS of the present invention thus include phytase, cellulase, lactase, lipase, protease, catalase, xylanase, beta-glucanase, mannanase, amylase, amidase, epoxide hydrolase, esterase, phospholipase, transaminase, amine oxidase, cellobiohydrolase, ammonia lyase, or any combination thereof. In one embodiment of the invention, an FDDS is provided as defined herein before, comprising a digestive enzyme selected from the group consisting of lactases.

An exemplary digestive enzyme that may particularly benefit from incorporation in an FDDS system according to the invention is acid lactase. Acid lactase is a beta-galactosidase enzyme that converts lactose into glucose and galactose. The enzyme is originally derived from Aspergillus oryzae and it is active at a low pH making it particularly attractive to apply the enzyme to the stomach. The enzyme can also be recombinantly produced according to the art. Tolerase™ L (DSM) for example is the commercial product name for the Aspergillus oryzae acid lactase enzyme which is produced in an Aspergillus niger microorganism.

Another exemplary digestive enzyme that may particularly benefit from incorporation in an FDDS system according to the invention is a protease, more preferably a prolyl endoprotease. Prolyl endoprotease, also known as prolyl endopeptidase, is an enzyme that cleaves peptide bonds at the C-terminal side of proline residues. The commercially available Aspergillus niger prolyl endoprotease (Tolerase™ G from DSM) degrades gluten molecules more effectively than digestive enzyme supplements currently available.

In another embodiment of the invention, the FDDS comprises a protein or peptide that exerts pharmacological effects locally. The formulation of such peptide or protein drugs in the form of the present FDDS may be beneficial in particular as it facilitates a constant supply of the protein or peptide to the site of action without the need for frequent administration, allowing therapeutically effective dosages to be delivered by once-a-day administration. As will be understood by those skilled in the art, in case of such locally acting peptide or protein drugs further protective measures may be applied, such as inclusion of the peptide/protein in nano-capsules so that the peptide/protein are protected from any negative influences of the environment during transit from the stomach to the site of action. The FDDS of the invention has been found to be very suitable to accommodate such variations.

In another embodiment of the invention, the FDDS comprises a protein or peptide that exerts pharmacological effects systemically. The formulation of such peptide or protein drugs in the form of the present FDDS may be beneficial in particular as it facilitates a constant supply of the protein or peptide at the site of absorption without the need for frequent administration. As will be understood by those skilled in the art, in case of such systemically acting peptide or protein drugs further protective measures may be applied, such as inclusion of the peptide/protein in nano-capsules, chemical modification (such as PEGylation), inclusion of protease inhibitors, etc., to protect them from the negative influences of the environment during transit from the stomach to the site of absorption. The FDDS of the invention has been found to be very suitable to accommodate such variations. Peptide drugs that may accordingly be suitable for inclusion in the FDDS include, but are not limited to, the peptidyl hormones activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B, insulin, luteinizing hormone (LH), luteinizing hormone-releasing hormone (LHRH), α-melanocyte-stimulating hormone, β-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, vasoactive intestinal peptide (VIP), and vasopressin. Other peptidyl drugs are the cytokines, e.g., colony stimulating factor 4, heparin binding neurotrophic factor (HBNF), interferon-α, interferon α-2a, interferon α-2b, interferon α-n3, interferon-β, etc., interleukin-1, interleukin-2, interleukin-3, interleukin-4, interleukin-5, interleukin-6, etc., tumor necrosis factor, tumor necrosis factor-α, granuloycte colony-stimulating factor (G-CSF), granulocyte-macrophage colony-stimulating factor (GM-CSF), macrophage colony-stimulating factor, midkine (MD), and thymopoietin. Still other peptidyl drugs that can be advantageously delivered using the present systems include endorphins (e.g. dermorphin, dynorphin, α-endorphin, β-endorphin, γ-endorphin, σ-endorphin, [Leu5] enkephalin, [Met5] enkephalin, substance P), kinins (e.g., bradykinin, potentiator B, bradykinin potentiator C, kallidin), LHRH analogues (e.g., buserelin, deslorelin, fertirelin, goserelin, histrelin, leuprolide, lutrelin, nafarelin, tryptorelin), and the coagulation factors, such as a 1-antitrypsin, a2-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, and thrombomodulin.

The coating layer comprises a coating excipient in combination with the peptide/protein. The coating excipient enables the formation of a stable, uniform layer surrounding the hollow particle. The coating excipient typically functions in the control of the release of the peptide or protein from the coating layer as well as to provide the integrity of the coating layer, resistance to the penetration of water/gastric juices, etc.

Typically, in accordance with an embodiment of the invention, the at least one coating excipient is a water-swellable polymer. Typically, by “water swellable polymer” is meant a polymer that does not readily dissolve in water (or does not dissolve in water at all) but interacts with water to cause the polymer to increase in volume. Polymers that may suitably be incorporated in the FDDS of this invention as the at least one coating excipient include polymers that are non-toxic and that swell in a dimensionally unrestricted manner upon submerging in water and/or (simulated) gastric fluid at a temperature within the range of 20-37° C.

Typically, in accordance with an embodiment of the invention, the at least one coating excipient is a hydrophilic polymer. In the present context, the term “hydrophilic” describes that the polymer has hydrophilic portions that typically are electrically polarized and capable of forming hydrogen bonds with water molecules, enabling it to dissolve more readily in water than in oil or other “non-polar” solvents.

Typically, in accordance with an embodiment of the invention, the at least one coating excipient is a polymer that hydrates to form a hydrogel when placed (submerged) in water and/or (simulated) gastric fluid at a temperature within the range of 20-37° C. Hydration, as used in this context, typically means that the polymer interacts with water so as to result in the association of water molecules with polymer groups and/or in water filling up voids within the polymer network. Typically such phenomena result in the formation of a hydrogel. Preferably the coating excipient is a quickly hydratable polymer.

Typically, in accordance with an embodiment of the invention, the at least one coating excipient forms a hydrogel when placed (submerged) in water and/or (simulated) gastric fluid at a temperature within the range of 20-37° C. The term hydrogel, as used in this context, typically refers to a visco-elastic material having an aqueous phase with an interlaced three-dimensional polymeric network.

Typically, in accordance with an embodiment of the invention, the at least one coating excipient is a polymer giving an apparent viscosity at 2% weight in water at 20° C. within the range of 1500-10000 mPa·s, preferably within the range of 2000-7500 mPa·s, more preferably within the range of 2500-5000 mPa·s.

In accordance with an embodiment of the invention, the coating excipient is non-toxic, typically meaning that it can be used in accordance with the invention without causing harmful effects to a human or animal body after ingestion of an FDDS. More preferably the coating excipient has a GRAS status.

Examples of polymers that may suitably be incorporated as the at least one coating excipient in the FDDS of the invention include: cellulose polymers and their derivatives, especially cellulose ethers, such as hydroxypropyl methylcellulose (including hydroxypropyl methylcellulose (HPMC) E3, HPMC E5, HPMC E6, HPMC E15, HPMC E4M, HPMC E10M, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, and HPMC P550; ethylcellulose polymers (such as Ethocel 4, Ethocel 7, Ethocel 10, Ethocel 14, and Ethocel 20); copovidone (vinylpyrrolidone-vinyl acetate copolymer 60/40), polyvinyl acetate, polyvinyl caprolactam-polyvinyl acetate-polyethylene glycol graft copolymer (SOLUPLUS®), methacrylate/methacrylic free acid copolymers (such as Eudragit L100-55, Eudragit L100, and Eudragit S100); polyethylene glycols (such as polyethylene glycol (PEG) 400, PEG 600, PEG 1450, PEG 3350, PEG 4000, PEG 6000, and PEG 8000); polaxamers (such as poloxamer 124, poloxamer 188, poloxamer 237, poloxamer 338, and poloxamer 407) carbomer; poly (2-ethyl-2-oxazoline); poly(ethyleneimine); polyurethane hydrogels; crosslinked polyacrylic acids and their derivatives, polysaccharides and their derivatives, including alginate, pectin, guar gum, dextrans, carrageenan, gellan, xanthan gum, chitosan, maltodextrins, starches, etc.; polyalkylene oxides; polyethylene glycols; poly(vinyl alcohol); maleic anhydride copolymers; polyvinylpyrolidones, such as polyvinylpyrolidone (PVP) K17, PVP K25, PVP K30, and PVP K90); and copolymers of the polymers listed above, including block copolymers and graft polymers, such as PLURONIC® and TECTONICS®, which are polyethylene oxide-polypropylene oxide block copolymers.

Cellulose polymers are particularly preferred in the context of the invention, in particular water-soluble and/or hydrophilic cellulose polymers. Examples of suitable cellulose polymers include, but are not limited to: hydroxyalkyl methyl celluloses such as hydroxypropyl methyl cellulose, hydroxybutyl methyl cellulose, hydroxyethyl methyl cellulose, hydroxymethyl methyl cellulose and hydroxyethylpropyl methyl cellulose; carboxyalkyl methylcelluloses such as carboxypropyl methyl cellulose, carboxybutyl methyl cellulose, carboxyethyl methyl cellulose, carboxymethyl methyl cellulose and carboxyethylpropyl methyl cellulose; hydroxyalkyl celluloses such as hydroxypropyl cellulose, hydroxybutyl cellulose, hydroxyethyl cellulose, hydroxymethyl cellulose and hydroxyethylpropyl cellulose; alkyl celluloses such as propyl cellulose, butyl cellulose, ethyl cellulose, methyl cellulose; and carboxyalkyl celluloses such as carboxypropyl cellulose, carboxybutyl cellulose, carboxyethyl cellulose, carboxymethyl cellulose and carboxyethylpropyl cellulose. Cellulose and cellulose ether derivative polymers may be of any length or combination of lengths. Moreover, the ranges of percent of substitutions may vary to ranges up to about 100%. In molecules comprising two or more different substituting groups, the percentage substitution for each group is independent of the other groups. The coatings of the present FDDS may comprise a single polymer type of cellulose or cellulose ether derivative, or may comprise a combination of one or more of cellulose and cellulose ether derivatives.

Particularly preferred examples of polymers that can be incorporated as the at least one coating excipient in the FDDS of the invention include cellulose ethers, in particular hydroxypropyl methyl cellulose (HPMC), hydroxypropyl cellulose (HPC), hydroxyethyl cellulose (HEC), methyl cellulose (MC), carboxymethyl cellulose (CMC), hydroxymethyl cellulose (HMC) and mixtures thereof.

Particularly preferred examples of polymers that can be incorporated as the at least one coating excipient in the FDDS of the invention include hydroxypropylmethyl cellulose. A family of HPMC products are available commercially from the Dow Chemical Company (under the trade designation Methocel™). Such commercial HPMC products are often referred to with the suffix E, F, J or K. The number following the letter suffix refers to viscosity in millipascal-seconds (mPa·s) measured at a 2% concentration in water at 20° C. A “C” after the number refer to “hundred”; an “M” refers to “thousand”. Suffix thereafter are additional identifiers, e.g. “P” refers to “Premium”, “LV” refers to “Low Viscosity”.

Particularly preferred examples of polymers that can be incorporated as the at least one coating excipient in the FDDS of the invention include low viscosity HPMC. In some embodiment it is preferred that the HPMC has a viscosity within the range of about 1500-10000 mPa·s, preferably within the range of 2000-7500 mPa·s, more preferably within the range of 2500-5000 mPa·s as determined as a 2% by weight aqueous solution of the HPMC at 20° C., using a Ubbelohde tube viscometer.

Particularly preferred examples of polymers that can be incorporated as the at least one coating excipient in the FDDS of the invention include HPMC K4M and HPMC E4M as the at least one coating excipient, most preferably said at least one coating excipient is HPMC E4M.

The coating layer may suitably contain additional ingredients. In one embodiment of the invention, the coating layer comprises one or more release modifying agents. The addition of such release modifying agents affects the rate of release of the peptide or protein from the coating. Suitable examples of such release modifying agents include starches; pregelatinized starches; stearate, preferably magnesium stearate and combinations thereof. The present inventors, in particular, have established, that these release modifying agents can suitably be used in combination with cellulose polymers and their derivatives to manipulate the release rate/behaviour, allowing therapeutically effective dosages to be delivered by once-a-day administration.

In an embodiment of the invention a floating drug delivery system (FDDS) is provided, wherein the coating surrounding the hollow particle comprises: 10-90 wt. %, based on the total weight of the coating, of one or more proteins and/or peptides; 10-90 wt. % of one or more water swellable polymers, preferably a cellulose polymer, such as hydroxypropyl methylcellulose; and 0-20 wt. % of a release modifying agent, preferably starch and/or magnesium stearate. In an embodiment of the invention a floating drug delivery system (FDDS) is provided, wherein the coating surrounding the hollow particle comprises 30-85 wt. %, based on the total weight of the coating, of one or more proteins and/or peptides; 15-70 wt. % of one or more water swellable polymers, preferably a cellulose polymer, such as hydroxypropyl methylcellulose; and 1-15 wt. % of a release modifying agent, preferably starch and/or magnesium stearate. In an embodiment of the invention a floating drug delivery system (FDDS) is provided wherein the coating surrounding the hollow particle comprises 40-80 wt. %, based on the total weight of the coating, of one or more proteins and/or peptides; 20-60 wt. % of one or more water swellable polymers, preferably a cellulose polymer, such as hydroxypropy Imethylcellulose; and; 3-12 wt. % of a release modifying agent preferably starch and/or magnesium stearate. In an embodiment of the invention a floating drug delivery system (FDDS) is provided, wherein the coating surrounding the hollow particle comprises 50-75 wt. %, based on the total weight of the coating, of one or more proteins and/or peptides; 25-50 wt. % of one or more water swellable polymers, preferably a cellulose polymer, such as hydroxypropylmethylcellulose; and; 5-10 wt. % of a release modifying agent preferably starch and/or magnesium stearate. In particularly preferred embodiments the water-swellable polymer is HPMC and the release modifying agent is magnesium stearate.

In another embodiment, the hollow particle is surrounded by a subcoating. The term “subcoating” refers to one or more layers applied in between the hollow particle and the protein and/or peptide containing coating layer.

In a preferred embodiment, the subcoating is a low pH resistant subcoating, meaning that the coating remains virtually intact at low pH and becomes water-soluble at higher pH values. Preferably, the subcoating comprises a low pH resistant coating material that becomes soluble at a pH value of 3.0 or higher, at a pH value of 3.5 or higher, at a pH value of 4.0 or higher, at a pH value of 4.5 or higher, at a pH value of 5.0 or higher or at a pH value of 5.5 or higher.

In a preferred embodiment of the invention the subcoating comprises or consists of one or more acid functional polymers such as hydroxypropylmethyl cellulose phthalate of varying grades (and also as an aqueous dispersion), methacrylate based polymers (e.g. Eudragit) and hydroxypropylmethyl cellulose acetate succinate, and other similar, pharmaceutically acceptable materials such as hydroxypropylmethyl cellulose acetate succinate, cellulose acetate succinate; cellulose acetate phthalate, hydroxypropylmethyl cellulose acetate succinate, cellulose acetate trimellitate, hydroxypropyl methyl cellulose phthalate, hydroxypropyl methyl cellulose acetate succinate, starch acetate phthalate, polyvinyl acetate phthalate, carboxymethyl cellulose, methyl cellulose phthalate, methyl cellulose succinate, methyl cellulose phthalate succinate, methyl cellulose phthalic acid half ester, and ethyl cellulose succinate. In a preferred embodiment of the invention the subcoating comprises or consists of hydroxypropyl methylcellulose phthalate. Such coating materials are commercially available. As an example, a low pH resistant coating material that becomes soluble at a pH of approximately 5.5 is commercially available under the trade name ‘HP-55’ from Shin-Etsu Chemical Co., Ltd. The application of this subcoating may be advantageous to prevent early degradation of the hollow particle under the influence of gastric fluid diffusing through the coating layer.

In another embodiment of the invention, the subcoating may comprise a hydrophilic cellulose derivative. A non-limitative list of suitable hydrophilic cellulose derivatives comprise of HPMC (hydroxypropylmethylcellulose also known as hypromellose), HPC (hydroxypropylcellulose), MC (methylcellulose), HEC (hydroxyethylcellulose), CMC (carboxymethylcellulose) and sodium-CMC. Preferably, the hydrophilic cellulose derivative is HPMC and even more preferably HPMC 606. The application of a subcoating comprising any of these materials is believed to improve the adherence of further coating layers.

In another embodiment of the invention, the hollow particle, preferably a capsule, is bordered by a wall comprising a pH resistant material. Preferably, the wall comprises an enteric material. Even more preferably, the wall comprises a low pH resistant material. Examples of such capsules include DRcaps™ and enTRinsic™ drug delivery technology capsules (Capsugel).

In another embodiment of the invention, an FDDS is provided comprising a further outer coating layer that does not contain protein or peptide. The use of an outer coating layer allows for accurate programming of active ingredient release.

In one embodiment, one or more of the coating(s) and subcoating(s) may comprise one or more additives having a beneficial or otherwise desired effect on a property of the coating. Useful additives include a plasticizer, a stabiliser, a pH adjuster, a GI motility adjuster, a viscosity adjuster, a diagnostic agent, an imaging agent, an expansion agent, a surfactant, and mixtures thereof.

In one embodiment, one or more of the coating(s) and subcoatings may comprise a plasticizer. The group of plasticizers contains, but is not limited to, materials such as polyethylene glycol (e.g. PEG6000), triethyl citrate, diethyl citrate, diethyl phthalate, dibutyl phthalate, tributyl citrate, and triacetin. The quantity of plasticiser included will be apparent to those skilled in the art.

In a particularly preferred embodiment of the invention, a floating drug delivery system (FDDS) as defined herein is provided, said FDDS comprising:
(a) a gelatin capsule;
(b) a subcoating comprising hydroxypropylmethylcellulose phthalate;
(c) a coating comprising 50-75 wt. %, based on the total weight of the coating, of one or more proteins and/or peptides; 25-50 wt. % of HPMC; and 5-10 wt. % of magnesium stearate.

In a particularly preferred embodiment of the invention, a floating drug delivery system (FDDS) as defined herein is provided, said FDDS comprising:
(a) a gelatin capsule;
(b) a first subcoating comprising hydroxypropylmethylcellulose and polyethylene glycol;
(c) a second subcoating comprising a hydroxypropylmethylcellulose phthalate that is soluble in water at a pH level of 5.5. or higher;
(d) a coating comprising 50-75 wt. %, based on the total weight of the coating, of one or more proteins and/or peptides; 25-50 wt. % of HPMC; and 5-10 wt. % of magnesium stearate.

As will be illustrated in the examples here below, the FDDS of the present invention can be loaded with relatively high amounts of protein and/or peptide. Depending on the target subject and/or dosage regimen, suitable dosage forms of the FDDS can be developed. In one embodiment, the FDDS comprises a particle (capsule) having a hollow, gas-filled core bordered by a wall of at least one aqueous soluble, erodible, disintegrating or degradable polymer, said wall being surrounded by a coating comprising 10 mg to 10 gram of the peptide or protein. Preferably, the coating comprises 20 to 8000 mg of the peptide or protein, more preferably 25 to 5000 mg, such as 20-1000 mg, 50-500 mg or 1000-2500 mg. Preferred examples of the FDDS of the invention contain the peptide or protein in a total amount of 100 mg, 150 mg, 200 mg, 250 mg, 300 mg, 400 mg, 500 mg or 600 mg or in a total amount within a range having any two of the recited values as the respective end-points.

As will be understood, floating dosage forms rely on their ability to float on gastric fluid. Gastric fluid has a density close to that of water, which is 1.004 g/ml. Therefore, for the system to remain afloat, the overall density of the system must be less than 1 g/ml. In one embodiment, a drug delivery system according to the invention has a density of less than 0.95 g/cm³. Lower densities, such as less than 0.9 g/cm³, more preferably less than 0.8 g/cm³ are of course preferred. In a specific aspect, the density is less than 0.7 g/cm³.

In a preferred embodiment a floating drug delivery system is provided that, upon administration to a subject to be treated, is capable of remaining in the stomach for a period extending over at least 2, at least 3, at least 4, at least 5, at least 6 hours, at least 8 hours or at least 10 hours typically in the fasted state. In an embodiment the FDDS is capable of remaining in the stomach for a period extending over at least 12 or at least 24 hours, typically in the fasted state. Furthermore, in a preferred embodiment of the invention an FDDS is provided that, upon administration to a subject to be treated, is capable of releasing protein and/or peptide to the gastrointestinal tract (GIT) (stomach and proximal small intestine) for a period extending over at least 2, at least 3, at least 4, at least 5 or at least 6 hours, typically in the fasted state. In an embodiment the FDDS is capable of releasing enzyme and/or peptide to the GIT for a period extending over at least 12 or at least 24 hours, typically in the fasted state. Preferably, a floating drug delivery system is provided according to the invention where the release of the protein and/or peptide is continuous for at least 2, for at least 3, for at least 4, for at least 5, for at least 6, for at least 7, for at least 8, for at least 9, for at least 10, for at least 11, for at least 12 or for at least 24 hours. More preferably, the continuous release of protein and/or peptide is linear over a period of at least 2, at least 3, at least 4, at least 5, at least 6, at least 7, at least 8, at least 9, at least 10, at least 11, at least 12 or at least 24 hours. Furthermore, in a preferred embodiment of the invention an FDDS is provided that, in a standard in vitro test in a so called USP dissolution apparatus, is capable of releasing protein and/or peptide from the delivery system in a so called slow release profile. Such a release profile is preferably characterized by a release of less than 45% of the total active ingredient content after 1 hour and/or the release of more than 30% and less than 75% after 3 hours and/or the release of less than 80% after 6 hours. In an alternative embodiment the release profile is characterized by the release of less than 35% of the total active ingredient content after 1 hour and/or the release of more than 30% and less than 75% after 5 hours and/or the release of more than 80% of the total active ingredient content after 10 hours. In an alternative embodiment the release profile is characterized by the release of less than 25% of the total active ingredient content after 1 hour and/or the release of more than 30% and less than 75% after 12 hours and/or the release of more than 80% of the total active ingredient content after 24 hours.

Unless specified otherwise in this document, in vitro testing of the FDDS system is carried out in a so called USP dissolution apparatus II. With the dissolution medium (500 to 1000 ml) at a temperature of 37° C. and a rotational speed of the paddle of 50 to 75 RPM. For investigating the release profile or floating capacity of the gastro-retentive systems, simulated gastric fluid of the following composition is used: 11.2 g citric acid monohydrate (C₆H₈O₇·H₂O) and 16.6 g Disodiumhydrogenphosphate dihydrate (Na₂HPO₄·2H₂O) in water (1000 ml); pH=4.6. Protein and peptide concentrations in the dissolution medium can be determined by any suitable analytical method, like ultraviolet absorption, colorimetric assay or HPLC analysis. It will be understood that in the case the protein is an enzyme, the activity of the enzyme can be assessed by any suitable assay known in the art.

In a preferred embodiment of the invention an FDDS is provided, which remains buoyant on the gastric fluid upon administration, typically to achieve the afore-defined goals. Usually the buoyancy is characterized by the floating time (h) and/or buoyancy AUC (mg h). In a preferred embodiment of the invention a floating delivery system is provided having a floating time of at least 2, at least 3, at least 4, at least 5 or at least 6 hours when tested in vitro in the USP dissolution apparatus II. In an embodiment an FDDS is provided having a floating time of at least 12 or at least 24 hours when tested in vitro in the USP dissolution apparatus II.

Another aspect of the invention concerns a method for providing a quantity of FDDS' as defined herein, said method comprising the steps of:
(a) providing a quantity of hollow particles, preferably capsules, made of at least one aqueous soluble, erodible, disintegrating or degradable material, which may optionally contain one or more subcoatings as defined elsewhere herein;
(b) providing a first liquid coating composition comprising at least one protein and/or peptide in a solvent and a second liquid coating composition comprising one or more coating materials in a solvent;
(c) producing a coating layer on the hollow particle by simultaneously spraying the first and second liquid coating compositions onto the hollow particles while allowing the solvents to evaporate such that a coating layer is formed on the hollow particles.

Step (a) preferably entails the manufacture of a conventional air-filled capsule according to well-established methods. The capsule can be a two-part conventional capsule as well as a single unit air filled capsule. If the application of one or more subcoatings is desired these can typically be applied by conventional coating methods. The preferred materials for these optional subcoatings, as defined herein, are conventionally applied as coatings in the development and production of pharmaceutical formulations and the person skilled in the art, accordingly, is well aware of suitable techniques to use in the context of the present invention.

It will be understood that for step (b) a skilled person will be able to choose the type(s) and relative amount(s) of the components to obtain a liquid (sprayable) coating.

The first liquid coating composition typically comprises a solution or dispersion of the peptide(s) and/or protein(s) in water or an aqueous solvent. The concentrations and relative amount of the peptide/protein in the coating composition may vary. In general, the coating dispersion will contain between about 1 and 50 wt. % of protein and/or peptide based on the total weight of the dispersion or solution. In preferred embodiments of the invention the first coating composition comprises between 1 and 30 wt %, preferably between 5 and 25 wt %, even more preferably between 10 and 20 wt % of protein and/or peptide, based on the total weight of the liquid composition. The first coating composition may typically comprise further components, particularly agents that aid in the stabilization of the peptides and/or proteins, such as pH adjusting agents, buffering agents, salts, etc.

The second liquid coating composition typically comprises a solution or dispersion of the coating materials and other optional ingredients, such as the release modifying agents, in an aqueous or organic solvent. Suitable solvents typically include water, alcohols, acetone and mixtures thereof. The solvent should, on the one hand be suitable to produce a sprayable solution or homogeneous dispersion of the coating material, while, on the other hand, it should not degrade the peptide/protein during the spray coating process. In accordance with the invention, particularly good results can be obtained with mixtures of acetone and water, e.g. in ratio's within the range of 1/1-20/1, 5/1-17/1 or 10/1-15/1. In preferred embodiments of the invention the second coating composition comprises between 1 and 15 wt %, preferably between 2 and 10 wt %, even more preferably between 3 and 8 wt % of coating material, based on the total weight of the liquid composition

Step (c) can suitably be performed with a conventional spray drying apparatus, most preferably as a fluid-bed spray-drying apparatus or a coating pan spraying apparatus, that can be equipped with two spraying nozzles in order to simultaneously spray the first and second liquid coating compositions. A suitable example of an apparatus that can be used to perform the process is the Soldilab 2 unit (from Bosch®).

In the process of the invention, the spray drying apparatus is typically operated so as to keep the temperature of the first liquid coating composition below a pre-determined maximum at all times. As will be understood by those skilled in the art, the protein and/or peptide contained in said first liquid coating composition typically is prone to degradation under the influence of high temperature and/or the chemicals needed to apply the coating excipient. The present inventors made the surprising discovery that by producing the coating by spraying two liquids simultaneously it is possible to minimize degradative processes, especially to minimize the time that the protein and/or peptide is effectively exposed to high temperatures and/or degradative chemicals. With this knowledge, the person skilled in the art will be able to determine suitable ways to operate the process based on routine optimization of the relevant parameters, such as inlet air temperature, outlet temperatures, spraying-rates, spray air pressure, etc.

In a particularly preferred embodiment of the invention, step (c) is performed in such a manner that the temperature of the first liquid coating composition during spraying does not exceed 50° C., preferably it does not exceed 47° C., more preferably it does not exceed 45° C., more preferably it does not exceed 43° C. more preferably it does not exceed 42° C., more preferably it does not exceed 41° C. and most preferably it does not exceed 40° C. In another preferred embodiment of the invention, step (c) is performed in such a manner that the temperature of the capsules on which the coating layer is growing does not exceed the above-cited temperature. Furthermore, during step (c) the spraying rate of the two spraying nozzles spraying the first and second liquid coating compositions are chosen such that the total amounts of first and second liquid coating composition needed for a given FDDS coating composition are sprayed within the same time frame. Another aspect of the invention relates to the use of a floating drug delivery system according to the invention to deliver a protein and/or peptide to the stomach or upper intestinal tract.

In another aspect of the invention, a floating drug delivery system as provided herein is advantageously used for the treatment or prophylaxis of a disease, for example in a method comprising administering to a customer and/or patient in need thereof a composition comprising a floating drug delivery system (FDDS) according to the invention. It will be understood that an FDDS of the invention, as with other floating systems, works optimal if the stomach of the subject receiving the FDDS is at least partially filled with gastric fluid. Therefore, it is preferred that the subject is a non-fasted subject. In case the subject is a fasted subject, the method comprises administering to the subject an oral floating drug delivery system (FDDS) together with a sufficient amount of fluid, e.g. an amount of water of at least 100 ml, preferably at least 200 ml.

In one aspect, the invention provides a method for treating, preventing, lessening the symptoms of and/or lessening the risk of developing a disease affecting the stomach or upper part of the intestinal tract, comprising administering to a customer and/or patient in need thereof a composition comprising a floating drug delivery system (FDDS) according to the invention, and wherein the protein and/or peptide is useful and effective in the treatment of the disease. In another aspect, the invention provides a method for treating, preventing, lessening the symptoms of and/or lessening the risk of developing a disease, comprising oral systemic drug administration, and wherein the protein and/or peptide is absorbed into the systemic circulation from only a limited part of the intestinal tract.

Also encompassed is a floating drug delivery system (FDDS) according to the invention for use in the treatment of, prevention of, lessening of the symptoms of and/or lessening the risk of developing a disease, preferably wherein the protein and/or peptide is useful in the topical treatment of the disease. Preferably, said disease is located in the stomach or upper intestinal tract. In an embodiment of the invention, the protein and/or peptide is a digestive enzyme and the FDDS of the invention is used and/or intended for use in a method of treating, preventing, lessening the symptoms of and/or lessening the risk of developing a condition or pathology of the digestive tract. In an embodiment of the invention, the protein and/or peptide is a digestive enzyme and the FDDS of the invention is used and/or intended for use in a method of treating, preventing, lessening the symptoms of and/or lessening the risk of developing a condition associated with the inability of a subject to digest components of a normal diet. In an embodiment of the invention, the protein and/or peptide is a Tolerase™ G and the FDDS of the invention is used and/or intended for use in a method of treating, preventing, lessening the symptoms of and/or lessening the risk of developing a condition associated with the inability of a subject to digest gluten. In an embodiment of the invention, the protein and/or peptide is a Tolerase™ L and the FDDS of the invention is used and/or intended for use in a method of treating, preventing, lessening the symptoms of and/or lessening the risk of developing a condition associated with the inability of a subject to digest lactose.

In another aspect, the invention relates to a floating drug delivery system (FDDS) according to the invention for use in the treatment of, prevention of, lessening of the symptoms of and/or lessening the risk of developing a disease, wherein the protein and/or peptide is absorbed into the systemic circulation from only a limited part of the intestinal tract.

A FDDS of the invention is particularly useful for delivering a therapeutic agent to the stomach or upper intestinal tract of a customer and/or patient and/or for enhancing the gastric retention of an agent in the stomach of a customer and/or patient, the method comprising oral administration to the customer and/or patient of a composition comprising a floating drug delivery system (FDDS), wherein a coating comprising the therapeutic agent is coated onto the surface of a solid particle, preferably a capsule, said particle having a hollow, gas-filled core bordered by a wall of at least one aqueous soluble, erodible, disintegrating or degradable material, typically a polymer. Preferably, said agent is a protein, a peptide or an enzyme or a mixture of different proteins, peptides or enzymes or any combination thereof.

Also encompassed is a method of enhancing the gastrointestinal absorption of a protein and/or peptide which is absorbed into the systemic circulation over only a limited part of the small intestine of a customer and/or patient, the method comprising oral administration to the customer and/or patient of the protein and/or peptide being incorporated in a FDSS as provided herein.

Unless otherwise indicated each embodiment as described herein may be combined with another embodiment as described herein. As will be understood by those skilled in the art, the principal features of this invention can be employed in the various aspects and embodiments without departing from the scope of the invention. More, in particular, it is contemplated that any feature discussed in this specification can be implemented with respect to any of the methods, compositions and uses of the invention, and vice versa.

It will also be understood that the term “protein” can be replaced with enzyme in any of the embodiments according to the invention provided herein.

Furthermore, for a proper understanding of this invention and its various embodiments it should be understood that in this document and the appending claims, the verb “to comprise” is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each embodiment as identified herein may be combined together unless otherwise indicated.

The following examples describe various new and useful embodiments of the present invention. It will be understood that particular embodiments described herein are shown by way of illustration and not as limitations of the invention. Those skilled in the art will recognize, or be able to ascertain using no more than routine experimentation, numerous equivalents to the specific procedures described herein. Such equivalents are considered to be within the scope of this invention and are covered by the claims.

### EXAMPLES

### Materials and Equipment

Aspergillus oryzae acid lactase (in these examples referred to as “Lactase”) was obtained from DSM (sold as Tolerase™ L), HPMC 606 and HPMC E4M from Dow, magnesium stearate from Genfarma, PEG 6000 from Merck and HP-55 from Shinetsu.

In all experiments demineralized water was used.

Aspergillus oryzae lactase was purchased as a 15.6% dry matter solution with a lactase activity of 24050 ALU/g. Enzyme activity was assessed using ONPG spectrophotometry according to the manufacturer's instructions as provided in the product sheet.

### Coating Method

The Aspergillus oryzae acid lactase containing coating was created by spraying the lactase containing solution and the acetone based HPMC spraying suspension in parallel through different spray nozzles, in order to minimize the wet contact time between lactase and the acetone solvent from the polymer suspension. This was done using two spray pumps so that it was possible to spray both solutions within the same time frame. The lactase solution and HPMC suspension were sprayed onto the surface of the capsules rotating in a small container under a heated air stream (the operating conditions are summarized in table for each example) until the required amount of drug-polymer mixture as determined by weight analysis was sprayed on the capsules.

The different surface area of the capsule sizes in the below examples was taken into consideration and the amount of the spraying liquids (or the number of capsules) adapted in order to obtain a comparable coating thickness for the different capsule sizes.

### Example 1: Preparation of Lactase Containing FDDS Without Subcoating (FDDS1)

Gelatin capsules size x were coated with lactase coating to provide a FDDS exemplary of one of the embodiments of the invention. The FDDS prepared according to this example is referred to in this and other examples as FDDS1. A lactase concentration of 320 mg/FDDS1 capsule is achieved.

Coating was performed using the Solidlab 2 fluid bed system equipped with a two nozzle bottom spray system using the reduction insert (working volume of 2 liter) with the following setup and ingredients:

### Application of Lactase Coating to Gelatin Capsules

| Liquid | Air cap | | Process | |
|---|---|---|---|---|
| tip size | size | Tube diameter | filters | Inlet air |
| [mm] | [mm] | [mm] | type | conditioning |
| 1.2 | 2.3 | 1.6 × 1.6 (enzyme | no | adsorption drier |
| | | solution); 3.2 × 1.6 | filters | (ca. 2 g/kg |
| | | (HPMC solution) | | residual moisture) |

### Solids

| Ingredient | Amount [number] | Percentage (w/w) |
|---|---|---|
| Gelatine capsules size 4 | 1220 | 100 |

### Spraying Solution—HPMC-E4M

| Ingredient | Amount [g] | Percentage (w/w) |
|---|---|---|
| HPMC E4M | 99 | 5.6 |
| Mg-Stearat | 4.9 | 0.3 |
| Acetone | 1500 | 85.5 |
| Water | 150 | 8.6 |
| Total: | 1754 | |

### Spraying Solution—Lactase

| Ingredient | Amount [g] | Percentage (w/w) |
|---|---|---|
| Lactase solution | 2503 | 100 |
| (solid content 15.6%) | (390.4 g Lactase) | |
| Total: | 2503 | |

### Process Data During the Spraying Step (Steady State)

| | |
|---|---|
| Inlet air volume [m³/h] | 80-125 |
| Inlet air temperature [° C.] | 50-60 |
| Outlet temperature [° C.] | 32-35 |
| Spray rate [g/min] | 2 × 12 |
| Inlet air humidity [g/kg] | 3-10 |
| Outlet air humidity [g/kg] | 8-11 |
| Spray air pressure [bar] | 0.6 |
| Microclimate pressure [bar] | 0.04 |

### Example 2: Preparation of Lactase Containing FDDS With Subcoating (FDDS2)

Gelatin capsules size 3 were subcoated with a HMPC 606 layer to close the gap between the two parts of the capsule, followed by an enteric layer of HP-66 to provide subcoated capsules. These capsules were further coated with lactase coating to provide a FDDS exemplary of one of the embodiments of the invention. The FDDS prepared according to this example is referred to in this and other examples as FDDS2. A lactase concentration of 490 mg/FDDS2 capsule is achieved.

Coating was performed using the Solidlab 2 fluid bed system equipped with a two nozzle bottom spray system using the reduction insert (working volume of 2 liter) with the following setup and ingredients:

### Application of Subcoating to Gelatin Capsules

| Liquid | Air cap | | Process | |
|---|---|---|---|---|
| tip size | size | Tube diameter | filters | Inlet air |
| [mm] | [mm] | [mm] | type | conditioning |
| 1.0 | 2.3 | 3.2 × 1.6 | no | only cooler |
| | | | filters | (ca. 8 g/kg |
| | | | | residual moisture) |

### Solids

| Ingredient | Amount [number] | Percentage (w/w) |
|---|---|---|
| Gelatine capsules size 3 | 951 (550 mL) | 100 |

### Spraying Solution—HPMC-Subcoating

| Ingredient | Amount [g] | Percentage (w/w) |
|---|---|---|
| HPMC 606 | 28.7 | 6.4 |
| PEG 6000 | 6.8 | 1.5 |
| Ethanol | 332 | 73.6 |
| Water | 83.6 | 18.5 |
| Total: | 451 | |

### Spraying Solution—HP-55-Subcoating

| Ingredient | Amount [g] | Percentage (w/w) |
|---|---|---|
| HP-55 | 57.6 | 6.0 |
| Ethanol | 767 | 79.8 |
| Water | 135.4 | 14.2 |
| Total: | 960 | |

### Process Data During the Spraying Step (HPMC-Part)

| | |
|---|---|
| Inlet air volume [m³/h] | 50-60 |
| Inlet air temperature [° C.] | 50-55 |
| Outlet temperature [° C.] | 28-30 |
| Spray rate [g/min] | 10 |
| Inlet air humidity [g/kg] | 10 |
| Outlet air humidity [g/kg] | 12 |
| Spray air pressure [bar] | 0.4 |
| Microclimate pressure [bar] | 0.01 |

### Process Data During the Spraying Step (HP-55-Part)

| | |
|---|---|
| Inlet air volume [m³/h] | 60-80 |
| Inlet air temperature [° C.] | 45 |
| Outlet temperature [° C.] | 30 |
| Spray rate [g/min] | 12 |
| Inlet air humidity [g/kg] | 11 |
| Outlet air humidity [g/kg] | 12 |
| Spray air pressure [bar] | 0.5 |
| Microclimate pressure [bar] | 0.02 |

### Application of Lactase Coating to Subcoated Capsules

| Liquid | Air | | Process | |
|---|---|---|---|---|
| tip size | cap size | Tube diameter | filters | Inlet air |
| [mm] | [mm] | [mm] | type | conditioning |
| 1.2 | 2.3 | 1.6 × 1.6 (enzyme | no | adsorption drier |
| | | solution); 3.2 × 1.6 | filters | (ca. 2 g/kg |
| | | (HPMC solution) | | residual moisture) |

### Solids

| | Amount | Percentage |
|---|---|---|
| Ingredient | [number] | (w/w) |
| subcoated capsules | 916 | 100 |

### Spraying Solution—HPMC-E4M

| | | Percentage |
|---|---|---|
| Ingredient | Amount [g] | (w/w) |
| HPMC E4M | 96.48 | 3.8 |
| Mg-Stearat | 6.76 | 0.3 |
| Acetone | 2192.5 | 86.3 |
| Water | 243.6 | 9.6 |
| Total: | 2539.3 | |

### Spraying Solution—Lactase

| | | Percentage |
|---|---|---|
| Ingredient | Amount [g] | (w/w) |
| Lactase solution (solid | 2443.1 (449.5 g | 100 |
| content 18.4%) | Lactase) | |
| Total: | 2443.1 | |

### Process Data During the Spraying Step (Steady State)

| | |
|---|---|
| Inlet air volume [m³/h] | 80-125 |
| Inlet air temperature [° C.] | 50-60 |
| Outlet temperature [° C.] | 32-35 |
| Spray rate [g/min] | 2 × 12 |
| Inlet air humidity [g/kg] | 3-10 |
| Outlet air humidity [g/kg] | 8-11 |
| Spray air pressure [bar] | 0.6 |
| Microclimate pressure [bar] | 0.04 |

### Example 3: Preparation of Lactase Containing FDDS With Subcoating (FDDS3)

Gelatin capsules size 4 were subcoated with a HMPC 606 layer to close the gap between the two parts of the capsule, followed by an enteric layer of HP-66 to provide subcoated capsule. These capsules were further coated with lactase coating to provide a FDDS exemplary of one of the embodiments of the invention. The FDDS prepared according to this example is referred to in this and other examples as FDDS3. A lactase concentration of 599 mg/FDDS3 capsule is achieved.

Coating was performed using the Solidlab 2 fluid bed system equipped with a two nozzle bottom spray system using the reduction insert (working volume of 2 liter) with the following setup and ingredients:

### Application of Subcoating to Gelatin Capsules

| | | Tube | Process | |
|---|---|---|---|---|
| Liquid tip | Air cap | diameter | filters | Inlet air |
| size [mm] | size [mm] | [mm] | type | conditioning |
| 1.0 | 2.3 | 3.2 × 1.6 | no filters | only cooler (ca. 8 g/kg |
| | | | | residual moisture) |

### Solids

| | Amount | Percentage |
|---|---|---|
| Ingredient | [number] | (w/w) |
| Gelatine capsules size 4 | 1190 (550 mL) | 100 |

### Spraying Solution—HPMC-Subcoating

| | | Percentage |
|---|---|---|
| Ingredient | Amount [g] | (w/w) |
| HPMC 606 | 29.5 | 6.4 |
| PEG 6000 | 6.7 | 1.5 |
| Ethanol | 341 | 73.6 |
| Water | 86 | 18.5 |
| Total: | 463 | |

### Spraying Solution—HP-55-Subcoating

| | | Percentage |
|---|---|---|
| Ingredient | Amount [g] | (w/w) |
| HP-55 | 58.9 | 6.0 |
| Ethanol | 784 | 79.8 |
| Water | 139 | 14.2 |
| Total: | 982 | |

### Process Data During the Spraying Step (HPMC-Part)

| | |
|---|---|
| Inlet air volume [m³/h] | 40-64 |
| Inlet air temperature [° C.] | 45-55 |
| Outlet temperature [° C.] | 28-33 |
| Spray rate [g/min] | 9 |
| Inlet air humidity [g/kg] | 8 |
| Outlet air humidity [g/kg] | 8 |
| Spray air pressure [bar] | 0.3 |
| Microclimate pressure [bar] | 0.03 |

### Process Data During the Spraying Step (HP-55-Part)

| | |
|---|---|
| Inlet air volume [m³/h] | 64-82 |
| Inlet air temperature [° C.] | 45-55 |
| Outlet temperature [° C.] | 32-35 |
| Spray rate [g/min] | 12 |
| Inlet air humidity [g/kg] | 8 |
| Outlet air humidity [g/kg] | 8 |
| Spray air pressure [bar] | 0.5 |
| Microclimate pressure [bar] | 0.01 |

### Application of Lactase Coating to Subcoated Capsules

| Liquid | Air cap | | Process | |
|---|---|---|---|---|
| tip size | size | | filters | Inlet air |
| [mm] | [mm] | Tube diameter [mm] | type | conditioning |
| 1.2 | 2.3 | 1.6 × 1.6 (enzyme | no | adsorption drier |
| | | solution); 3.2 × 1.6 | filters | (ca. 2 g/kg |
| | | (HPMC solution) | | residual moisture) |

### Solids

| | Amount | Percentage |
|---|---|---|
| Ingredient | [number] | (w/w) |
| Capsules of 16-35-V2-1 | 750 | 100 |

### Spraying Solution—HPMC-E4M

| | | Percentage |
|---|---|---|
| Ingredient | Amount [g] | (w/w) |
| HPMC E4M | 96.48 | 3.8 |
| Mg-Stearat | 6.76 | 0.3 |
| Acetone | 2192.5 | 86.3 |
| Water | 243.6 | 9.6 |
| Total: | 2539.3 | |

### Spraying Solution—Lactase

| | | Percentage |
|---|---|---|
| Ingredient | Amount [g] | (w/w) |
| Lactase solution (solid | 2443.1 (449.5 g | 100 |
| content 18.4%) | Lactase) | |
| Total: | 2443.1 | |

### Process Data During the Spraying Step (Steady State)

| | |
|---|---|
| Inlet air volume [m³/h] | 80-125 |
| Inlet air temperature [° C.] | 50-60 |
| Outlet temperature [° C.] | 32-35 |
| Spray rate [g/min] | 2 × 12 |
| Inlet air humidity [g/kg] | 3-10 |
| Outlet air humidity [g/kg] | 8-11 |
| Spray air pressure [bar] | 0.6 |
| Microclimate pressure [bar] | 0.04 |

### Example 4: SEM Imaging of FDDS Capsules

### FDDS1

FIG. 3 shows a SEM image of a cross section of the FDDS1 capsule showing the wide enzyme layer and no subcoating layer.

### FDDS2

FIG. 2 shows a SEM image of a cross section of the FDDS2 capsule showing the wide enzyme layer and the two pre-coating layers. The two pre-coating layers are nicely homogenous with a good integrity. The very wide enzyme layer displays some cracks and porosity. Presumably this is due to the fact that a HPMC-E4M suspension of solid material was used in the drug layering step (as opposed to real solutions in the precoating steps). Thus, the solid particles never had the chance to form a continuous film. Nevertheless, this does not affect the desired sustained release, presumably as the HPMC swells as soon as coming in contact with water (or gastric fluid).

### FDDS3

FIG. 1 shows a SEM image of a cross section of the FDDS3 capsule showing the wide enzyme layer and the two pre-coating layers. The two pre-coating layers are nicely homogenous with a good integrity. The very wide enzyme layer displays some cracks and porosity. Presumably this is due to the fact that a HPMC-E4M suspension of solid material was used in the drug layering step (as opposed to real solutions in the precoating steps). Thus, the solid particles never had the chance to form a continuous film. Nevertheless, this does not affect the desired sustained release, presumably as the HPMC swells as soon as coming in contact with water (or gastric fluid).

### Example 5: Lactase Containing FDDS Release Testing

### Dissolution Method

Release experiments were performed in a Sotax dissolution apparatus. The apparatus was loaded with 1000 ml buffer solution, prepared by adding 11.2 g citric acid monohydrate (C₆H₈O₇·H₂O) and 16.6 g Disodiumhydrogenphosphate dihydrate (Na₂HPO₄·2H₂O) to water and adding water to volume. The mixture was stirred until homogenous. The pH of the resulting citric acid buffer was measured and, if required, adjust to (PH=4.6). The resulting dissolution medium is stirred at 50 rpm and 37° C. and one capsule was added to the dissolution medium via the opening on top of the dissolution device. Samples (2 ml) were taken during the dissolution experiment at 0, 1, 2, 3, 4, 5, 6, 7, 8, (9) and 24 hours.

### Lactase Concentration Determination

From the above described samples the activity of dissolved Lactase was determined using ONPG spectrophotometry according to the manufacturer's instructions as provided in the product sheet. Subsequently, the activity of the samples was compared with a calibration curve to determine the lactase concentration. The calibration curve was created by measuring the activity of a series of Lactase solutions prepared using lactase from the same batch as the lactase used to prepare the FDDS of the present invention.

### Results

FIG. 5 shows the Lactase release profiles of the capsules of the present invention and provides a comparison with the release profile of the Tetesept tablet, a commercially available tablet containing Lactase (16.000 FCC units per Tablet) for use as a medicament for humans who are lactose intolerant.

The Tetesept tablet rapidly sunk to the bottom or the dissolution apparatus, suggesting bad gastro-retentivity.

In conclusion, all of the FDDS systems according to the invention allows loading high dosages, show a continuous release of the enzyme, with a linear release profile for at least up to 9 hours and display long-lasting floating behavior, indicating satisfactory gastro-retentivity and thus allowing therapeutically effective dosages to be delivered by once-a-day administration.

### Example 6

FIG. 4 shows the Lactase release profile in a volume of 1 l of three capsules prepared in a similar manner as the FDDS3 system described above; but with 435 mg Tolerase L per capsule (193200 ALU/g Tolerase L batch). The linear release profile is evident for at least 9 hours and an average yield of 87% is achieved.

### FIGURES

FIG. 1: A SEM image of a cross section of the FDDS3 capsule showing the wide enzyme layer and the two pre-coating layers.

FIG. 2: A SEM image of a cross section of the FDDS2 capsule showing the wide enzyme layer and the two pre-coating layers.

FIG. 3: A SEM image of a cross section of the FDDS1 capsule showing the wide enzyme layer.

FIG. 4: Release profile in ALU/ml for 3 capsules of batch FDDS3.

FIG. 5: Comparison of release profiles in mg/l of FDDS1, FDDS2, FDDS3 and the commercially available Tetesept tablet.

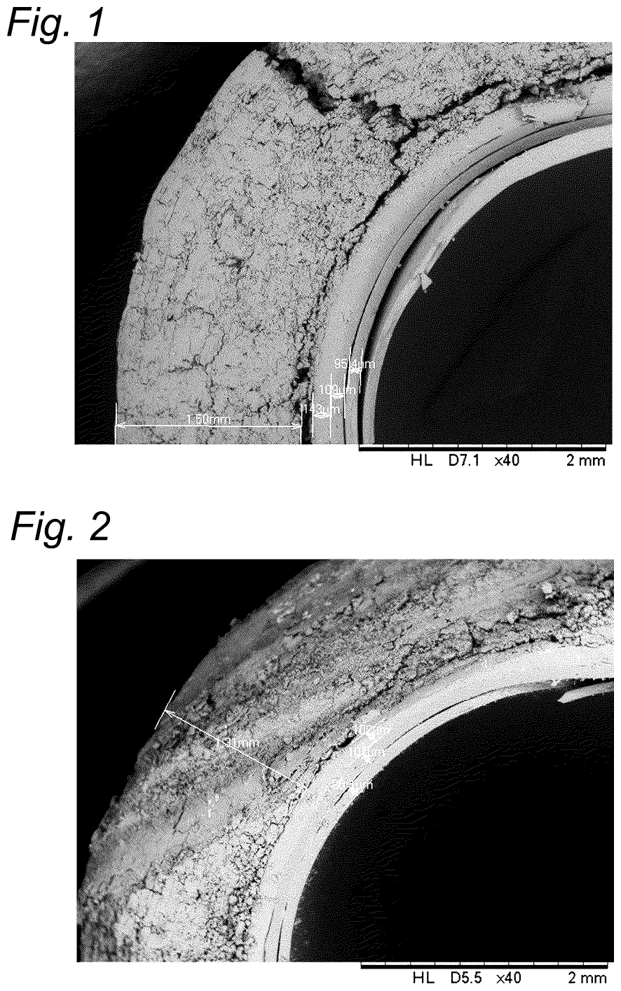

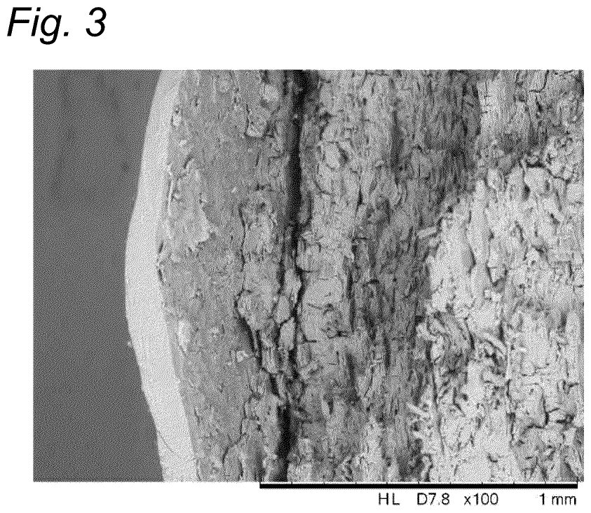

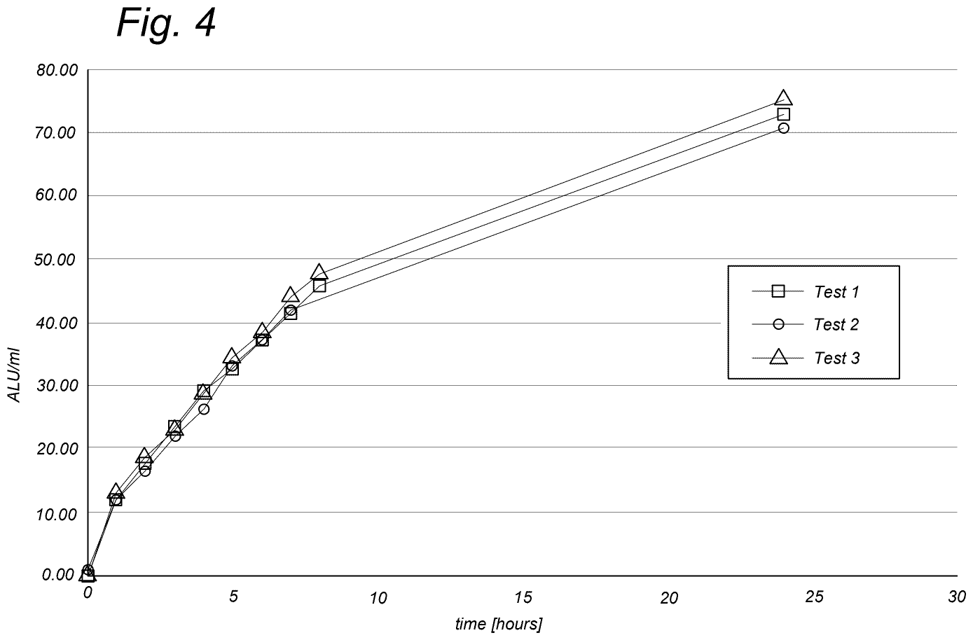

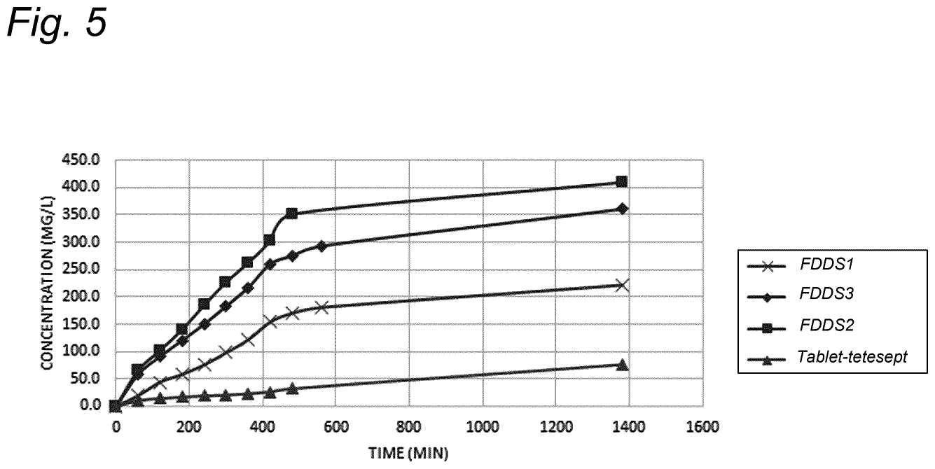

1. Floating drug delivery system (FDDS), said FDDS comprising
(i) a hollow particle, comprising a gas-filled and/or hollow core bordered by a wall of at least one material selected from the group of aqueous soluble, erodible, disintegrating and biodegradable materials, preferably a polymer; and
(ii) a coating surrounding said hollow particle comprising at least one protein and/or peptide in combination with at least one coating excipient.

2. Floating drug delivery system according to claim 1, wherein said hollow particle is a gelatin or vegetarian capsule.

3. Floating drug delivery system according to claim 1, wherein said coating comprises a polymer selected from the group consisting of hydroxyalkyl methyl celluloses such as hydroxypropyl methyl cellulose, hydroxybutyl methyl cellulose, hydroxyethyl methyl cellulose, hydroxymethyl methyl cellulose and hydroxyethylpropyl methyl cellulose; carboxyalkyl methylcelluloses such as carboxypropyl methyl cellulose, carboxybutyl methyl cellulose, carboxyethyl methyl cellulose, carboxymethyl methyl cellulose and carboxyethylpropyl methyl cellulose; hydroxyalkyl celluloses such as hydroxypropyl cellulose, hydroxybutyl cellulose, hydroxyethyl cellulose, hydroxymethyl cellulose and hydroxyethylpropyl cellulose; alkyl celluloses such as propyl cellulose, butyl cellulose, ethyl cellulose, methyl cellulose; and carboxyalkyl celluloses such as carboxypropyl cellulose, carboxybutyl cellulose, carboxyethyl cellulose, carboxymethyl cellulose and carboxyethylpropyl cellulose.

4. Floating drug delivery system according to claim 1, wherein said coating comprises hydroxypropylmethylcellulose (HPMC).

5. Floating drug delivery system according to claim 1, wherein said coating comprises a stearate salt or ester, preferably magnesium stearate.

6. Floating drug delivery system according to claim 1, comprising a subcoating in between the hollow particle and the peptide and/or protein containing coating layer, said subcoating comprising an enteric polymer.

7. Floating drug delivery system according to claim 7, wherein said enteric coating comprises hydroxypropylmethylcellulose phthalate (HPMCP) or HP-55, preferably HP-55.

8. Floating drug delivery system according to claim 1, wherein the peptide or protein is a digestive enzyme.

9. Floating drug delivery system according to claim 1, wherein the FDDS comprises a total dosage of the peptide and/or protein of at least 0.05 g, preferably 0.1 g, more preferably 0.2 g.

10. Floating drug delivery system according to claim 1, wherein the release of the protein and/or peptide is continuous and/or linear for at least 6 hours.

11. Floating drug delivery system according to claim 1, which is capable of remaining in the stomach for at least 6 hours and/or of releasing protein and/or peptide for at least 6 hours.

12. Floating drug delivery system according to claim 1, wherein said coating is a coating selected from the group consisting of coatings resistant to gastric juice, release-controlling coatings, and mixtures thereof.

13. Floating drug delivery system according to claim 1, having a density less than 0.95 g/cm3, preferably less than 0.9 g/cm3, more preferably less than 0.8 g/cm3 and even more preferred less than 0.7 g/cm3.

14. Floating drug delivery system according to claim 1, said FDDS comprising:
(a) a gelatin or vegetarian capsule;
(b) a first subcoating comprising hydroxypropylmethylcellulose and polyethylene glycol;
(c) a second subcoating comprising a hydroxypropylmethylcellulose phthalate that is soluble in water at a pH level of 5.5 or higher;
(d) a coating comprising 50-75 wt. %, based on the total weight of the coating, of one or more proteins and/or peptides; 25-50 wt. % of HPMC; and 5-10 wt. % of magnesium stearate.

15. Method for the production of providing a quantity of FDDS as defined in claim 1, said method comprising the steps of:
(a) providing a quantity of hollow particles, preferably capsules, made of at least one aqueous soluble, erodible, disintegrating or degradable material, which may optionally contain one or more subcoatings as defined herein;
(b) providing a first liquid coating composition comprising at least one protein and/or peptide in a solvent and a second liquid coating composition comprising one or more coating materials in a solvent;
(c) producing a coating layer on the hollow particle by simultaneously spraying the first and second liquid coating compositions onto the hollow particles while allowing the solvents to evaporate such that a coating layer is formed on the hollow particles.
